(19) United States
(12) Patent Application Publication
(10) Pub. No.: US 2011/0300067 A1
(43) Pub. Date: Dec. 8, 2011

(54) METHODS FOR INCREASING EFFICACY OF RADIOIMMUNOTHERAPY OF MELANOMA

(76) Inventors: Ekaterina Dadachova, Mahopac, NY (US); Arturo Casadevall, New Rochelle, NY (US)

(21) Appl. No.: 12/998,021

(22) PCT Filed: Sep. 2, 2009
(86) PCT No.: PCT/US09/04939

§ 371 Date: May 24, 2011

Related U.S. Application Data
(60) Provisional application No. 61/191,513, filed on Sep. 9, 2008.

(51) Int. Cl. A61K 51/10; A61N 5/00; A61K 51/08; A61P 35/00; A61P 37/04

(52) U.S. Cl. 424/1.49; 424/1.69; 600/3; 600/1

(57) ABSTRACT

Methods are disclosed for using agents and treatments that cause the release of melanin from melanin-containing melanomas to increase the efficacy of therapy and imaging of the melanomas with radiolabeled anti-melanin antibodies and peptides.

### CROSS-REFERENCE TO RELATED APPLICATION

This application claims the benefit of U.S. Provisional Patent Application No. 61/191,513, filed on Sep. 9, 2008, the content of which is hereby incorporated by reference.

### FIELD OF THE INVENTION

The present invention relates to methods of using agents and treatments that cause the release of melanin from melanin-containing melanomas to increase the efficacy of treatment of the melanomas and to improve the imaging of melanomas with radiolabeled anti-melanin antibodies and radiolabeled anti-melanin peptides.

### BACKGROUND OF THE INVENTION

Throughout this application various publications are referred to in parentheses. Full citations for these references may be found at the end of the specification immediately preceding the claims. The disclosures of these publications are hereby incorporated by reference in their entireties into the subject application to more fully describe the art to which the subject application pertains.

There is a clinical need for therapies for melanoma, which is among the few cancers with a rising incidence (Rigel, 1997). Malignant melanoma affects ˜40,000 new patients each year in the United States and an estimated 100,000 world-wide (Grin-Jorgensen et al. 1992, Liu and Soong, 1996). Melanoma is an important cause of cancer among young patients (30-50 years) which increases the economic importance of the disease. Although primary tumors that are localized to the skin can be successfully treated by surgical removal, a satisfactory treatment for patients with metastatic melanoma has not been introduced clinically (Sun and Schuchter, 2001). The median survival time of patients with metastatic melanoma is 8.5 months, with an estimated 5-year survival of 6% (Sun and Schuchter, 2001). There has been little change in these results over the past 25 years.

Targeted radionuclide therapy has evolved into an efficient modality for cancer patients in whom standard anti-neoplastic therapies have failed (Srivastava and Dadachova 2001). One type of targeted radionuclide therapy—radioimmunotherapy (RIT)—takes advantage of the specificity of the antigen-antibody interaction to deliver tumoricidal doses of radiation to target cells using radiolabeled antibodies (Milenic et al. 2004, Sharkey and Goldenberg, 2005). The clinical success of FDA-approved drugs such as ZEVALIN® and BEXXAR® (anti-CD20 monoclonal antibodies labeled with 90-Yttrium (⁹⁰Y) and 131-Iodine (¹³¹I), respectively) for the treatment of relapsed or refractory B-cell non-Hodgkin lymphoma (NHL) demonstrates the potential of RIT as an anti-neoplastic strategy.

Melanoma owes its name to the presence of the pigment melanin. Given that even amelanotic melanomas contain some melanin, this pigment presents a potential target for development of radionuclide therapy of metastatic melanoma. Historically, melanin was not considered a target for RIT because it is an intracellular pigment contained within organelles called melanosomes, which are outside the reach of a non-internalizing antibody. However, because melanomas are rapidly growing tumors, cell turnover releases melanin into the extracellular space where it can be targeted for delivery of cytotoxic radiation by radiolabeled melanin-binding antibodies. Experimental results have established the feasibility of targeting melanin released from dead melanoma cells in tumors with antibodies (Dadachova et al. 2004) and peptides (Dadachova et al. 2006, Howell et al. 2007) labeled with beta-emitting radionuclide 188-Rhenium (¹⁸⁸Re) (U.S. Pat. No. 7,402,385, United States Patent Application Publication No. 2006/0039858). Furthermore, this strategy is attractive because melanin in normal tissues is not accessible to the antibody by virtue of its intracellular location. Additional pre-clinical development of melanin-binding ¹⁸⁸Re-6D2 mAb, including pharmacokinetics, efficacy, and acute hematologic toxicity studies in a metastatic human melanoma model in mice (Dadachova et al. 2008) preceded to completed Phase I trial in patients with metastatic melanoma (Klein et al. 2008) that showed targeting of ¹⁸⁸Re-6D2 mAb to the tumors.

The present invention is directed to increasing the efficacy of the targeting of cytocidal radiation to the tumors using melanin-binding mAbs or peptides.

### SUMMARY OF THE INVENTION

The present invention is directed to methods for treating melanin-containing melanomas in a subject that comprise (a) administering to the subject an agent or treatment that releases melanin from melanoma cells, and (b) administering to the subject an amount of a radiolabeled anti-melanin antibody and/or radiolabeled anti-melanin peptide effective to treat the melanoma. The invention also provides methods for imaging melanin-containing melanomas in a subject that comprise (a) administering to the subject an agent or treatment that releases melanin from melanoma cells, and (b) administering to the subject an amount of a radiolabeled anti-melanin antibody and/or radiolabeled anti-melanin peptide effective to image the melanoma. The methods improve the efficacy of therapy and imaging of the melanoma by increased targeting of the radiolabeled anti-melanin antibody and/or peptide to melanin released from the tumor by the agent or treatment.

### BRIEF DESCRIPTION OF THE FIGURES

FIG. 1A-1C. Binding of ¹⁸⁸Re-11B11 mAb to fungal melanin and to MNT1 highly pigmented human melanoma cells: A) immunoreactivity of 11B11 as assessed by fungal melanin ELISA. Melanin-binding 6D2 mAb and irrelevant IgM 5C11 were used as positive and negative controls, respectively; B) binding to whole and osmotically lysed MNT1 cells; open squares—whole cells, closed squares—lysed cells; C) Scatchard analysis of ¹⁸⁸Re-11B11 mAb binding to MNT1 cells.

FIG. 2. Radioimmunotherapy of A2058 melanoma-bearing mice with ¹⁸⁸Re-11B11 melanin-binding mAb. Mice were given IP either 1 mCi ¹⁸⁸Re-11B11 mAb (hot mAb), 100 μg 11B11 mAb (cold mAb), or PBS. The change in tumors volume is shown with Vo being a tumor volume on the day of treatment, and Vt—tumor volume on the day of measurement.

FIG. 3A-3C. Histology of A2058 melanoma tumors from DTIC-treated mice: A) H&E-stained untreated A2058 tumor; B) melanin granules in necrotic tumor tissue of A2058 human metastatic melanoma-bearing mice treated for 5 days with DTIC. A finely granular very uniform golden brown pigment within the lesions, particularly at the junction of the necrotic and viable tissue which is assumed to be melanin. This pigment was not evident in viable cells except for scattered cells along the junction between viable-necrotic tumor; C) consecutive slides from DTIC-treated tumors stained with H&E (left panel), iron stain (middle panel) and melanin bleach (right panel) and viewed in polarized light. The absence of blue hemosiderin and hematin (middle panel) and disappearance of brown coloration of granules post-bleaching (right panel) confirmed the identity of the pigment released from the cells in DTIC-treated tumors as melanin.

FIG. 4A-4B. Comparison of the efficacy of chemotherapy alone, RIT alone, and combination of chemotherapy and RIT in mice with A2058 melanoma. A) change in tumor volume in mice control mice in comparison with mice given chemotherapy alone (50 mg/kg DTIC IP for 5 days), or RIT alone (1 mCi ¹⁸⁸Re-6D2 mAb), or combination of chemotherapy and RIT (50 mg/kg DTIC IP for 5 days followed by 1 mCi ¹⁸⁸Re-6D2 mAb). Vo—a tumor volume on the day of treatment, Vt—tumor volume on the day of measurement. Day 0 means the start of a therapy regiment for each particular group; B) H&E-stained tumor from RIT alone treated mouse showing the infiltration with inflammation-related cells.

FIG. 5A-5D. Coronal ¹⁸F-FDG microPET images of A2058 melanoma-bearing mice treated with chemotherapy or RIT. Mice were imaged on Days 0 and 7. White arrows point to the tumors. A) untreated mouse on Day 0; B) mouse treated with 1 mCi ¹⁸⁸Re-6D2 mAb on Day 1 and imaged on Day 7; C) untreated mouse on Day 7; D) mouse treated with 50 mg/kg DTIC IP for 5 days and imaged on Day 7.

### DETAILED DESCRIPTION OF THE INVENTION

The present invention is directed to a method for treating a melanin-containing melanoma in a subject that comprises (a) administering to the subject an agent or treatment that releases melanin from melanoma cells, and (b) then administering to the subject an amount of a radiolabeled anti-melanin antibody and/or radiolabeled anti-melanin peptide effective to treat the melanoma. The invention also provides a method for imaging a melanin-containing melanoma in a subject that comprises (a) administering to the subject an agent or treatment that releases melanin from melanoma cells, and (b) then administering to the subject an amount of a radiolabeled anti-melanin antibody and/or radiolabeled anti-melanin peptide effective to image the melanoma.

The melanin-containing melanoma can be a pigmented melanoma, a hypomelanotic melanoma, or an “amelanotic melanoma.” So-called “amelanotic melanomas” are generally hypomelanotic and contain small amounts of melanin (Busam et al. 2001; Cohen-Solal et al. 2002).

The term “treat” a melanoma means to eradicate the melanoma, to reduce the size of the melanoma, to stabilize the melanoma so that it does not increase in size, to reduce the further growth of the melanoma, or to prevent or reduce the spread of the melanoma.

The agent or treatment that releases melanin from melanoma cells can be, for example, one or more of 1) chemotherapy, e.g. using dacarbazine (5-(3,3-dimethyl-1-triazeno)imidazole-4-carboxamide, DTIC), which is a standard treatment for melanoma, or other melanocyte cytotoxic drugs, 2) external beam radiation therapy (EBRT), which is another standard treatment for melanoma, 3) brachytherapy, e.g. 90Y-seeds for use e.g. in ocular melanoma, 4) immunotherapy, e.g. antibody-toxin conjugates such as geldanamacine-HMW melanoma-associated antigen-binding monoclonal antibodies (mAbs), antibodies to melanoma cells that promote cell lysis, and radiolabeled or toxin-labeled antibodies to melanoma antigens, 5) biological therapy, e.g. therapy with interleukin IL-27 and other members of IL-6/IL-12 family, 6) cellular therapy, e.g. therapy with cytotoxic T cells that kill melanoma cells, 7) gene therapy, e.g. Bax mRNA therapy, and 8) radionuclide therapy such as 125I- or 131I-labeled iodobenzamides or astatinated with 211At methylene blue.

The preferred time period between administering the agent or treatment to the subject and administering the radiolabeled anti-melanin antibody and/or peptide can vary between several hours to several days, depending upon how quickly a particular agent or treatment causes release of melanin from the melanoma cells.

As used in the subject application, the term “antibody” encompasses whole antibodies, fragments of whole antibodies, chimeric antibodies, and humanized antibodies. Antibody fragments include, but are not limited to, F(ab′)₂ and Fab′ fragments. F(ab′)₂ is an antigen-binding fragment of an antibody molecule with deleted crystallizable fragment (Fc) region and preserved binding region. Fab′ is ½ of the F(ab′)₂ molecule possessing only ½ of the binding region. The term antibody is further meant to encompass polyclonal antibodies and monoclonal antibodies. The antibody can be an antibody generated against human melanin. A humanized antibody contains a constant region from a human antibody and an antigen binding region from a mouse antibody.

The antibody can be any of an IgA, IgD, IgE, IgG, or IgM antibody. The IgA antibody can be an IgA1 or an IgA2 antibody. The IgG antibody can be an IgG1, IgG2, IgG2a, IgG2b, IgG3 or IgG4 antibody. A combination of any of these antibodies can also be used. One consideration in selecting the type of antibody to be used is the desired serum half-life of the antibody. IgG has a serum half-life of 23 days, IgA 6 days, IgM 5 days, IgD 3 days, and IgE 2 days (Abbas et al. 2000). Another consideration is the size of the antibody. For example, the size of IgG is smaller than that of IgM allowing for greater penetration of IgG into tumors. IgA, IgG, and IgM are preferred antibodies.

The antibody can be 11B11 or 6D2. Preferably, the antibody is 6D2.

Melanin-binding peptides can also be used. Melanin-binding peptides have been described where the melanin-binding peptide is, for example, a decapeptide (Nosanchuk et al. 1999) or a heptapeptide (Howell et al. 2007). Different length peptides, or mixtures of different length peptides, can be used as well. Important structural characteristic of melanin-binding peptides are the presence of aromatic amino acids and overall positive charge.

A preferred melanin-binding decapeptide is 4B4 (YERKFWHGRH) (SEQ ID NO:1). Additional melanin-binding decapeptides include LHKLVRHGRW (SEQ ID NO:2), YLRRHTHVFW (SEQ ID NO:3), KKHSHYWVRY (SEQ ID NO:4), EFGTRHMRHR (SEQ ID NO:5), YRHHAHGGRG (SEQ ID NO:6), RKKWHGWTRW (SEQ ID NO:7), PKWRHGYTRF (SEQ ID NO:8), RHGTVKHARH (SEQ ID NO:9), RRHWHPPVQI (SEQ ID NO:10), EAYKRRWHWP (SEQ ID NO:11), RWPKRHLSGH (SEQ ID NO:12), SRVPFRHYHH (SEQ ID NO:13), RRPEHTKARW (SEQ ID NO:14), WRAFLPRWHA (SEQ ID NO:15), WNRGWRWWMG (SEQ ID NO:16), GFFWKWRIGR (SEQ ID NO:17) and HIRWKGHISW (SEQ ID NO:18). Preferred melanin-binding peptides comprise the amino acid motif X₁-X₂-X₃-X₄-H (SEQ ID NO:19), where X₁ and X₂ are positively charged amino acids, and X₃ and X₄ are positively charged amino acids and/or aromatic amino acids. Lysine (K or Lys), arginine (R or Arg), and histidine (H or His) are positively charged amino acids. Aromatic amino acids include histidine, phenylalanine (F or Phe), tyrosine (Y or Tyr), and tryptophan (W or Try).

Preferred melanin-binding heptapeptides include NPNWGPR (SEQ ID NO: 20), HTTHHRN (SEQ ID NO: 21) and TTHQFPF (SEQ ID NO: 22).

The choice of the particular radioisotope with which the antibody or peptide is labeled can be determined by the size of the melanoma to be treated and its localization in the body. Two characteristics are important in the choice of a radioisotope—emission range in the tissue and half-life. Alpha emitters, which have a short emission range in comparison to beta emitters, may be preferable for treatment of small melanomas that are disseminated in the body. Examples of alpha emitters include 213-Bismuth (half-life 46 minutes), 223-Radium (half-life 11.3 days), 224-Radium (half-life 3.7 days), 225-Radium (half-life 14.8 days), 225-Actinium (half-life 10 days), 212-Lead (half-life 10.6 hours), 212-Bismuth (half-life 60 minutes), 211-Astatine (half-life 7.2 hours), and 255-Fermium (half-life 20 hours). In a preferred embodiment, the alpha-emitting radioisotope is 213-Bismuth). ²¹³Bi emits a high LET α-particle with E=5.9 MeV with a path length in tissue of 50-80 μm. Theoretically a cell can be killed with one or two α-particle hits. ²¹³Bi is the only α-emitter that is currently available in generator form, which allows transportation of this isotope from the source to clinical centers within the United States and abroad.

Beta emitters, with their longer emission range, may be preferable for the treatment of large melanomas. Examples of beta emitters include 188-Rhenium (half-life 16.7 hours), 90-Yttrium (half-life 2.7 days), 32-Phosphorous (half-life 14.3 days), 47-Scandium (half-life 3.4 days), 67-Copper (half-life 62 hours), 64-Copper (half-life 13 hours), 77-Arsenic (half-life 38.8 hours), 89-Strontium (half-life 51 days), 105-Rhodium (half-life 35 hours), 109-Palladium (half-life 13 hours), 111-Silver (half-life 7.5 days), 131-Iodine (half-life 8 days), 177-Lutetium (half-life 6.7 days), 153-Samarium (half-life 46.7 hours), 159-Gadolinium (half-life 18.6 hours), 186-Rhenium (half-life 3.7 days), 166-Holmium (half-life 26.8 hours), 166-Dysprosium (half-life 81.6 hours), 140-Lantanum (half-life 40.3 hours), 194-Irridium (half-life 19 hours), 198-Gold (half-life 2.7 days), and 199-Gold (half-life 3.1 days). In a preferred embodiment, the beta-emitting radioisotope is 188-Rhenium. ¹⁸⁸Re is a high-energy β-emitter (Eₘₐₓ=2.12 MeV) that has recently emerged as an attractive therapeutic radionuclide in diverse therapeutic trials including cancer radioimmunotherapy, palliation of skeletal bone pain, and endovascular brachytherapy to prevent restenosis after angioplasty (Hoher et al. 2000, Knapp, 1998, Palmedo et al. 2000). ¹⁸⁸Re has the additional advantage that it emits γ-rays, which can be used for imaging studies. For the treatment of large melanomas or those in difficult to access sites deep in the body, longer-lived isotopes such as 90-Yttrium (half-life 2.7 days), 177-Lutetium (half-life 6.7 days) or 131-Iodine (half-life 8 days) may also be preferred.

Positron emitters can also be used, such as (half-life in parenthesis): ^{52m}Mn (21.1 min); ⁶²Cu (9.74 min); ⁶⁸Ga (68.1 min); ¹¹C (20 min); ⁸²Rb (1.27 min); ¹¹⁰In (1.15 h); ¹¹⁸Sb (3.5 min); ¹²²I (3.63 min); ¹⁸F (1.83 h); ^{34m}C1 (32.2 min); ³⁸K (7.64 min); ⁵¹Mn (46.2 min); ⁵²Mn (5.59 days); ⁵²Fe (8.28 h); ⁵⁵Co (17.5 h); ⁶¹Cu (3.41 h); ⁶⁴Cu (12.7 h); ⁷²As (1.08 days); ⁷⁵Br (1.62 h); ⁷⁶Br (16.2 h); ^{82m}Rb (6.47 h); ⁸³Sr (1.35 days); ⁸⁶Y (14.7 h); ⁸⁹Zr (3.27 days); ^{94m}TC (52.0 min); ¹²⁰I (1.35 h); ¹²⁴I (4.18 days). 64-Copper is a mixed positron, electron and Auger electron emitter.

Any of the radioisotopes, except alpha emitters, that are used for radioimmunotherapy can also be used at lower doses for radioimmunoimaging, for example a beta emitter, a positron emitter or an admixture of a beta emitter and a positron emitter. Preferred radioisotopes for use in radioimmunoimaging include 99m-Technetium, 111-Indium, 67-Gallium, 123-Iodine, 124-Iodine, 131-Iodine and 18-Fluorine. For imaging one can use a dose range of 1-30 mCi for diagnostic isotopes (e.g., 99m-Tc) and 1-10 mCi for therapeutic isotopes to avoid unnecessary dose to a patient.

Preferably, the radiolabeled anti-melanin antibody specifically binds to melanin. Preferably, the radiolabeled anti-melanin peptide specifically binds to melanin.

The invention further provides methods for treating melanoma that comprise administering to the subject anti-melanin antibodies and/or peptides radiolabeled with a plurality of different radioisotopes effective to treat the melanoma. Preferably, the radioisotopes are isotopes of a plurality of different elements. In a preferred embodiment, at least one radioisotope in the plurality of different radioisotopes is a long range emitter and at least one radioisotope is a short range emitter. Examples of long range emitters include beta emitters and positron emitters. Examples of short range emitters include alpha emitters. Positron emitters can also be intermediate range emitters depending on the energy of the positrons. In a preferred embodiment, the long-range emitter is a beta emitter and the short range emitter is an alpha emitter. Preferably, the beta emitter is 188-Rhenium. Preferably, the alpha emitter is 213-Bismuth. Combinations of different radioisotopes can be used, which include an admixture of any of an alpha emitter, a beta emitter, and a positron emitter, with physical half-lives from 30 minutes to 100 days. Preferably, the plurality of different radioisotopes is more effective in treating the tumor than a single radioisotope within the plurality of different radioisotopes, where the radiation dose of the single radioisotope is the same as the combined radiation dose of the plurality of different radioisotopes.

It is known from radioimmunotherapy studies of tumors that whole antibodies usually require from 1 to 3 days time in circulation to achieve maximum targeting. Although slow targeting may not impose a problem for radioisotopes with relatively long half-lives such as ¹⁸⁸Re (t_{1/2}=16.7 hours), faster delivery vehicles are needed for short-lived radioisotopes such as ²¹³Bi (t_{1/2}=46 min). The smaller melanin-binding peptides and F(ab′)₂ and Fab′ fragments provide much faster targeting, which matches the half-lives of short-lived radionuclides (Buchsbaum 2000, Saha 1997).

The subject can be a mammal. In different embodiments, the mammal is a mouse, a rat, a cat, a dog, a horse, a sheep, a cow, a steer, a bull, livestock, a primate, a monkey, or preferably a human.

The dose of the radioisotope can vary depending on the localization and size of the melanoma, the method of administration of radiolabeled antibody (local or systemic) and the decay scheme of the radioisotope. In order to calculate the doses that can treat the melanoma without radiotoxicity to vital organs, a diagnostic scan of the patient with the antibody radiolabeled with a diagnostic radioisotope or with a low activity therapeutic radioisotope can be performed prior to therapy, as is customary in nuclear medicine. The dosimetry calculations can be performed using the data from the diagnostic scan (Early and Sodee, 1995). In different embodiments, the dose of the radioisotope for RIT is about 1 mCi to about 1000 mCi.

Clinical data (Paganelli et al. 1999, Sgouros et al. 1999) indicate that fractionated doses of radiolabeled antibodies and peptides are more effective than single doses against tumors and are less radiotoxic to normal organs. Depending on the status of a patient and the effectiveness of the first treatment with RIT, the treatment can consist of one dose or several subsequent fractionated doses.

Preferably, the uptake of radiolabeled anti-melanin antibody or peptide in the melanoma is at least 10 times greater than in surrounding muscle or other tissue. Preferably, the radiolabeled anti-melanin antibody or peptide is not taken up by non-cancerous (i.e., normal or healthy) melanin-containing tissue, including, but not limited to, hair, eyes, skin, brain, spinal cord, and/or peripheral neurons.

Preferably, the combined procedure of (1) administering to the subject an agent or treatment that releases melanin from melanoma cells, and (2) then administering to the subject an amount of a radiolabeled anti-melanin antibody and/or radiolabeled anti-melanin peptide is more effective to treat and/or image the melanoma than either single procedure alone.

The invention provides for the use of an agent or treatment that releases melanin from melanoma cells for increasing the efficacy of radioimmunotherapy of melanoma and/or for improving the radioimmunoimaging of melanoma. The invention also provides for the use of an agent that releases melanin from melanoma cells for the preparation of a medicament for increasing the efficacy of radioimmunotherapy of melanoma and/or for improving the radioimmunoimaging of melanoma.

This invention will be better understood from the Experimental Details that follow. However, one skilled in the art will readily appreciate that the specific methods and results discussed are merely illustrative of the invention as described more fully in the claims which follow thereafter.

### EXPERIMENTAL DETAILS

### Materials and Methods

Antibodies and melanin ELISA. Fungal melanin binding mAbs 6D2 and 11B11, both of IgM isotype, were previously described in (Rosa et al. 2000a). MAb 6D2 was produced by Goodwin Biotechnology Inc. (Plantation, Fla.) and purified using a multicolumn purification system (Dadachova et al. 2008). Purity of the 6D2 from this process was >95% via HPLC-SEC. MAb 11B11 was obtained from supernatant made by growing the 11B11 hybridoma cells in standard DMEM with 5% FCS. The antibody was captured on a column using agarose beads with anti-mouse IgM (Sigma), eluted using acid and then neutralized to pH 7. The antibody concentration was determined by ELISA by comparison to a commercial standard.

For melanin ELISA Corning polystyrene medium binding plates were covered with 5×10⁵-5×10⁶ fungal melanin particles suspended in water and plated in each well. Plates were incubated at room temperature to allow the particles to dry and adhere to the surface. The plates were washed three times with TBS (20 mM Tris pH 7.2, 140 mM NaCl) after each incubation. Plates were blocked for 2 h at 37° C. with SuperBlock Blocking Buffer in PBS (Pierce, Rockville, Ill.). Antibodies were serially diluted in PBS (140 mM NaCl, 2.7 mM KCl, 1.5 mM KH₂PO₄, 8.5 mM Na₂HPO₄; pH7.4), starting with a 50 μg/mL concentration and incubated for 1.5 h at 37° C. After washing, a 1:100 dilution of goat anti-mouse IgM alkaline phosphatase-conjugated (Southern Biotechnologies Associates Inc., Birmingham, Ala.) was added to the wells and incubated for 1.5 h at 37° C. Antibody binding was detected by addition of p-nitrophenyl phosphate (Sigma Chemical Co.) solution in reaction buffer (1.0 mM MgCl₂ and 50 mM Na₂CO₃; pH9.8). After 30 min, solutions were transferred to a clear plate and measured at 405 nm with a μQuant™ Microplate Spectrophotometer (BioTek Instruments, Inc., Winooski, Vt., USA) (Rosa et al. 2000b).

Radioisotope and radiolabeling. ¹⁸⁸Re as sodium perrhenate Na¹⁸⁸ReO₄ was eluted from ¹⁸⁸W/¹⁸⁸Re generator (Oak Ridge National Laboratory, Oak Ridge, Tenn.). MAbs 6D2 and 11B11 were radiolabeled with ¹⁸⁸Re “directly” via generating —SH groups on mAbs with dithiothreitol as described in Dadechova et al. (2004).

Cells lines and in vitro binding experiments. MNT1 is a highly pigmented human melanoma cell line (a gift from Dr. V. Hearing, NIH) that was cultivated in MEM/20% FBS medium. The lightly pigmented melanoma cell line A2058 (American Type Culture Collection, Manassas, Va.) was grown in Dulbecco's MEM with 4 mM L-Glutamine, 4.5 g/L glucose, 1.5 g/L sodium bicarbonate, supplemented with 10% fetal bovine serum and 5% penicillin-streptomycin solution at 37° C. and 5% carbon dioxide. Both cell lines were harvested using 0.25% (w/v) Trypsin-EDTA solution. The cells were washed in serum-free Dulbecco's Modified Eagle's Medium before use.

For in vitro binding experiments 0.1 nM ¹⁸⁸Re-11B11 mAb was added to the increasing number of the whole or osmotically lysed MNT1 cells. After 1 hr incubation at 37° C., the activity in the tubes was measured in a gamma counter, the cells were collected by centrifugation and the pellets were counted again. Percentage binding to the cells was determined from the ratio of counts in the pellet to the counts in the tube. For Scatchard binding determinations, increasing amounts (0.053 nM to 0.256 nM) of ¹⁸⁸Re-11B11 mAb were added to osmotically lysed MNT1 cells (4×10⁶ cells per sample). Scatchard analysis was used to compute the mAb binding constant Kₐ to melanin and number of binding sites per cell as in Lindmo et al. (1984). The centrifuge tubes used in binding and Scatchard experiments were pre-blocked with 1% BSA to prevent non-specific protein binding.

Animal model and therapy studies. All animal studies were carried out in accordance with the guidelines of the Institutes for Animal Studies at the Albert Einstein College of Medicine. For RIT studies, 6-12 week-old female nude mice were implanted subcutaneously with 8×10⁶ A2058 human melanoma cells into the right flank and used for therapeutic experiments 12 days after tumor volumes were approximately 0.15 cm³ (0.02-0.4 cm³).

RIT with ¹⁸⁸Re-11B11. For investigating the ability of 11B11 mAb to deliver therapeutic doses of ¹⁸⁸Re to the tumors, the mice were randomized into three groups of five animals. The RIT group received intraperitoneally (IP) 1 mCi ¹⁸⁸Re-11B11 (100 μg) (“hot” mAb). The control groups received IP injections of either 100 μg unlabeled (“cold”) 11B11 or PBS. Mice were weighed and tumor volumes were measured immediately before administration of mAbs and every 3-4 days thereafter. Tumors were measured in three dimensions with calipers, and tumor volume was calculated by multiplying the product of the three perpendicular diameters by 0.5, assuming an elliptical geometry.

Combination of chemotherapy and RIT. For combined chemotherapy with dacarbazine and RIT of A2058 melanoma-bearing nude mice, a preliminary experiment was performed which was aimed at determining the tolerability of dacarbazine by tumor-bearing mice and the ability of dacarbazine treatment to release some melanin from the melanoma cells. For this purpose 10 mg/mL dacarbazine in citrate buffer with pH ˜5.5 was prepared by dissolving 100 mg dacarbazine (Sigma) in 9.5 mL citrate buffer, pH 3.9 and 0.5 mL 0.1 M HCl. Five mice with A2058 tumors were treated for 5 days IP with 50 mg/kg dacarbazine (1.1 mg/mouse). Three days after completion of DTIC treatment, two mice were sacrificed, their tumors removed, fixed in ethanol/buffered formalin, cut into 5 μm slices and analyzed histologically for the presence of melanin by staining with hematoxylin and eosin (H&E), iron staining or by melanin bleach. The slides were viewed under 400× magnification in polarized light. The remaining three mice were observed for their tumor size and body weight for 25 days.

For the experiment comparing the efficacy of chemotherapy, RIT and combination of chemotherapy, and RIT in A2058 melanoma-bearing mice, mice were inoculated with A2058 melanoma cells, randomized into groups of five after tumor volumes reached 0.15 cm³ (0.02-0.4 cm³). On Day 0 the treatment of groups #1 and 2 with 50 mg/kg DTIC for 5 consecutive days IP was initiated. Mice in group #3 received a single IP injection of 1 mCi ¹⁸⁸Re-6D2 mAb on Day 0. Twenty-four hrs after completion of pre-treatment with DTIC (Day 6), mice in group #2 received single IP dose of 1 mCi ¹⁸⁸Re-6D2 mAb. Group #4 was given PBS IP on Day 0. Mice were observed for their body weight and tumor size as described above.

MicroPositron Emission Tomography (microPET) of RIT or chemotherapy treated melanoma-bearing mice. To understand the effects of RIT with melanin-binding mAbs and chemotherapy with DTIC on A2058 tumor metabolism, microPET was performed of mice treated with DTIC or with RIT. For this purpose 6-7 mm in diameter tumor-bearing mice were fasted for 3 hrs, then placed in an anesthesia chamber with 1.5% isoflurane-oxygen mixture. Anesthesia continued through the IV injection, during a 1 hr uptake and through the imaging portion of the procedure. Each mouse was placed on a heating pad before scanning to maintain normal body temperature. Mice were injected via tail-vein with 11.1 to 14.8 MBq (300 to 400 μCi) ¹⁸F-FDG and 1 hr later imaged in an R4 microPET scanner manufactured by CTI Concorde. The Full Width at Half Maximum for the R4 is about 2.1 mm with a field of view of 120 mm and a depth of field of 78 mm. Images were acquired for 10 minutes with a lower level discrimination of 350 keV and upper level discrimination of 650 keV. The timing window was set to 6 nsec. All of the default settings were selected during the histogram process. Reconstruction was performed in OSEM2D (Ordered Subsets Expectation Maximization Two Dimension) reconstruction algorithm. Images were reconstructed in iterative reconstruction in a 128×128×64 (0.82×0.82×1.2 mm) pixel array. Data corrected for dead time counting losses, arc correction, random coincidences and the measured uniformity of detector responses (i.e. normalized) but not corrected for attenuation or scatter. The axial cutoff (Nyquist) in scatter settings was applied at 0.5. Twenty-four hrs after imaging, the mice were treated with either RIT (a single IP injection of 1 mCi ¹⁸⁸Re-6D2 mAb) or DTIC (50 mg/kg) for 5 consecutive days IP or given PBS IP. The mice were re-imaged one week after the initial PET scan.

Statistical analysis. The Wilcoxon rank sum test was used to compare tumor sizes between different treatment groups in therapy studies. Differences were considered statistically significant when P values were <0.05.

### Results

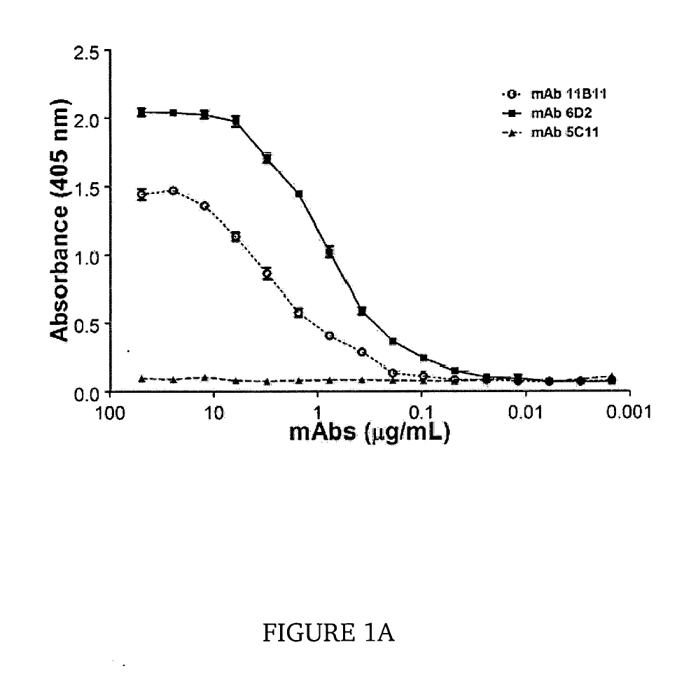

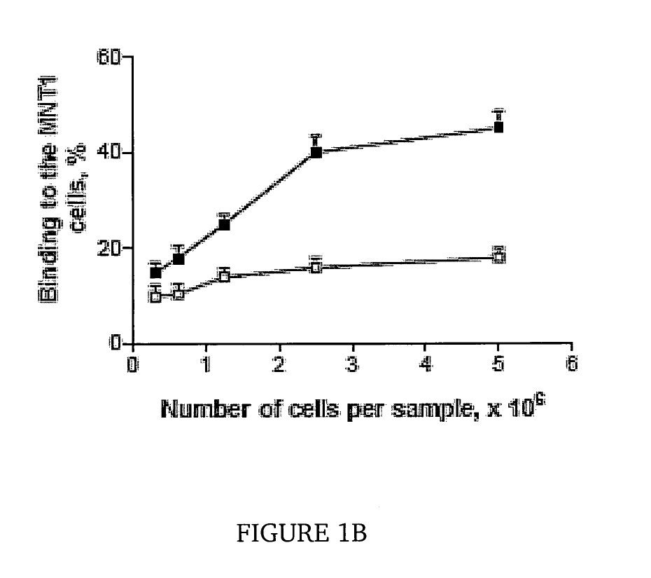

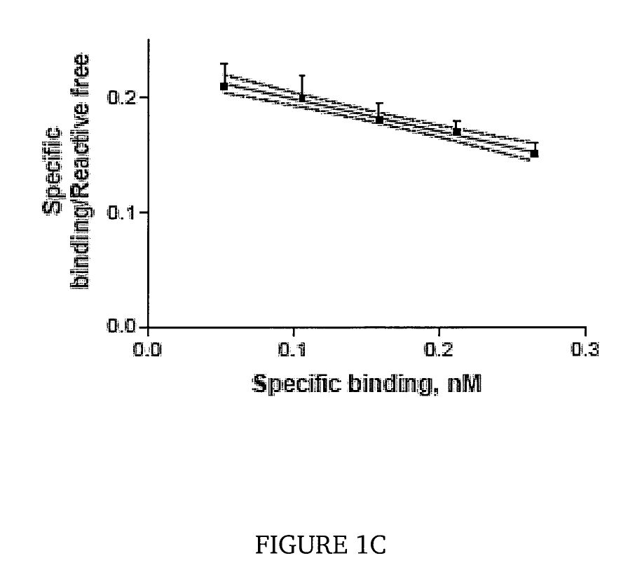

¹⁸⁸Re-11B11 showed high affinity binding constant for melanin. Melanin-binding ELISA demonstrated that 11B11 was binding to fungal melanin which confirmed its immunoreactivity (FIG. 1A). Binding of ¹⁸⁸Re-11B11 to MNT1 highly melanized cells was significantly enhanced by lysing the cells to release intracellular pigment consistent with the specificity of the mAb for melanin (FIG. 1B). Scatchard plot (FIG. 1C) revealed an affinity constant of 2.8×10⁸ M⁻¹ for 11B11 mAb and 1.2×10⁵ binding sites per lysed MNT1 cell. The affinity constant for ¹⁸⁸Re-11B11 was 1.5 times higher than for 6D2 mAb, which was previously determined to be 1.8×10⁸ M⁻¹ (Schweitzer et al. 2007). In contrast, the number of binding sites per cell for 11B11 was almost 3 times less in comparison with 3.1×10⁵ binding sites for 6D2 (Schweitzer et al. 2007), which might explain the somewhat lower binding of ¹⁸⁸Re-11B11 to MNT1 cells when compared with earlier data for ¹⁸⁸Re-6D2 (Dadachova et al. 2006a).

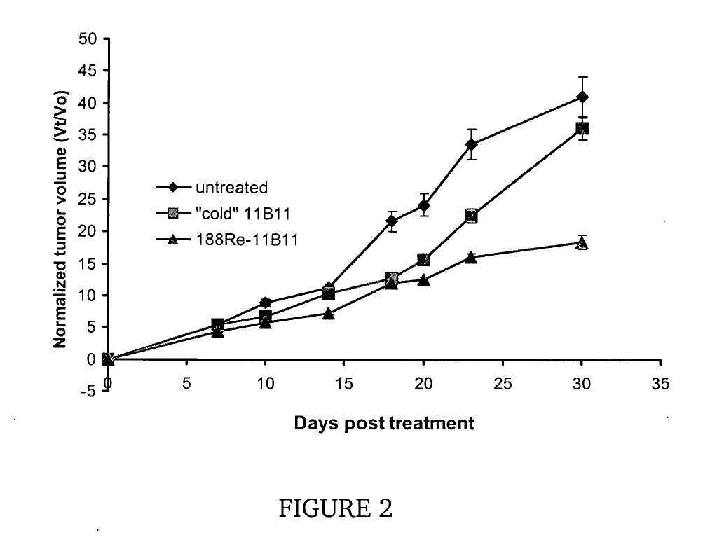

¹⁸⁸Re-11B11 was therapeutic in experimental human metastatic melanoma. To assess if the high binding affinity constant of ¹⁸⁸Re-11B11 would affect the therapeutic efficacy of this radiolabeled antibody, a RIT experiment was conducted in nude mice implanted with A2058 lightly pigmented human melanoma cells. Treatment of mice with 1 mCi ¹⁸⁸Re-11B11 resulted in significant inhibition of tumor growth in comparison with untreated controls or mice given “cold” 11B11 (p<0.05) (FIG. 2). “Cold” 11B11 impaired tumor growth from day 18 in comparison with untreated controls (p=0.04). RIT with ¹⁸⁸Re-11B11 was well tolerated and mice did not lose weight as a result of the treatment. The therapeutic results with ¹⁸⁸Re-11B11 were similar to those reported earlier with ¹⁸⁸Re-6D2 (Dadachova et al. 2008).

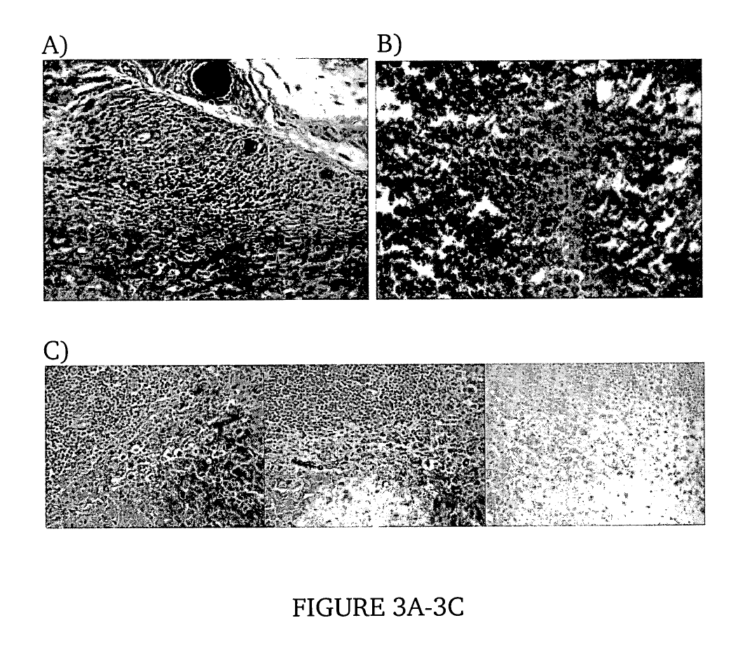

Pre-treatment of A2058 tumor-bearing mice with DTIC releases some melanin from the cells. Tumor bearing mice were treated with 50 mg/kg DTIC for 5 days to test the hypothesis that chemotherapy increases extracellular melanin for targeting by RIT since dying melanoma cells should release intracellular melanin. On histological evaluation of the tumors the melanin pigment was not evident in viable tumor cells except for occasional cells along the junction between viable-necrotic tumor (FIG. 3A), whereas tumors from DTIC-treated mice revealed a finely granular, uniform golden brown pigment within the lesions, particularly at the junction of the necrotic and viable tissue, which was assumed to be melanin (FIG. 3B). To prove that the brown pigment was extracellular melanin, the consecutive slides from DTIC-treated tumors were stained with H&E, iron stain and melanin bleach and viewed in polarized light. The absence of blue hemosiderin and hematin (FIG. 3C, middle panel) and disappearance of brown coloration of granules post-bleaching (FIG. 3C, right panel) confirmed the identity of the pigment released from the cells in DTIC-treated tumors as melanin.

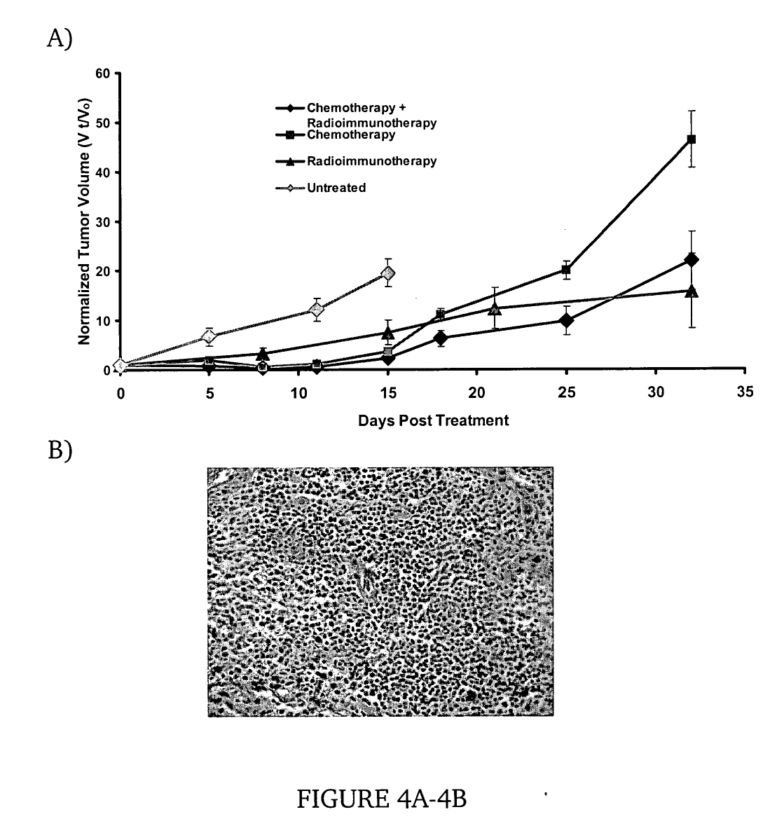

Combination chemotherapy and RIT was more effective in treating melanoma than chemotherapy or RIT alone. The exposure of melanin in the tumors after treatment with DTIC provided encouragement for conducting combination therapy studies in melanoma-bearing mice. Given the relative equivalence of mAbs 6D2 and 11B11 in binding and efficacy studies, this study was limited to ¹⁸⁸Re-6D2 since this mAb is currently in clinical trials. FIG. 4A shows the influence of the treatment on tumor progression in various groups. Tumors in control mice given only PBS grew aggressively such that by Day 15 all mice had to be sacrificed. During the early time points post-therapy (up to Day 15) both chemotherapy alone and chemotherapy plus RIT with ¹⁸⁸Re-6D2 were more effective in impairing tumor growth than RIT alone. However, from day 15 the tumor-growth trend changed such that the tumors in chemotherapy only group started to grow much faster than in either the combined treatment or RIT alone groups (p=0.02). The combined treatment was more effective in stabilizing the tumors than RIT alone up to Day 25 (p=0.03). By Day 27 the efficacy of combined treatment and RIT only equalized (p=0.06). The histological analysis of RIT-treated tumors at the completion of the study revealed significant infiltration of the tumors by inflammatory cells (FIG. 4B).

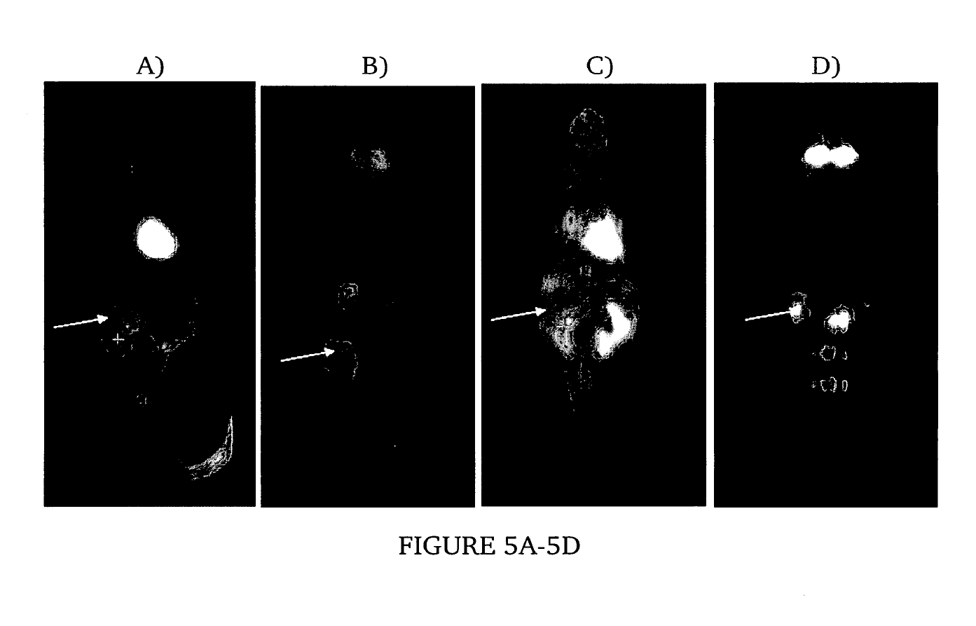

MicroPET of tumor-bearing mice before treatment showed pronounced ¹⁸F-FDG uptake in the tumors (FIG. 5A). The ¹⁸F-FDG uptake and the tumor sizes in untreated mice on the follow-up scans one week later increased significantly (FIG. 5B). When the effects of chemotherapy or RIT with ¹⁸⁸Re-6D2 on tumor metabolism were compared, the ¹⁸F-FDG uptake in RIT-treated tumors almost disappeared (FIG. 5C) whereas in chemotherapy-treated tumors it became less diffuse and concentrated in the center of the tumor (FIG. 5D).

### Discussion

The encouraging results of RIT with ¹⁸⁸Re-labeled melanin-binding mAb 6D2 in two different models of experimental melanoma (Dadachova et al. 2006a, 2008), followed by localization of ¹⁸⁸Re-6D2 in tumor sites in patients with metastatic melanoma (Klein et al. 2008) provided impetus to seek ways to improve the targeting of cytocidal radiation to the tumors using melanin-binding mAbs.

MAb 11B11 was compared to mAb 6D2, which is currently in clinical development. MAb 11B11 was generated against fungal melanin simultaneously with 6D2 mAb (Rosa et al. 2002a), and is attractive because its hybridoma line is quite stable and a high producer of immunoglobulin. Based on many chemical and structural similarities that are shared by fungal and mammalian melanins (Dadachova and Casadevall 2005) and on the ability of 6D2 to bind to both fungal and mammalian melanin (Dadachova et al. 2006a), 11B11 mAb was also expected to bind mammalian tumor melanin. Binding of ¹⁸⁸Re-11B11 to MNT1 highly melanized cells was melanin-specific as lysing of the cells which makes more melanin accessible for a melanin-binding mAb resulted in increased binding. Though the absolute binding of ¹⁸⁸Re-11B11 to melanoma cells was lower than for ¹⁸⁸Re-6D2, its therapeutic efficacy was very similar to ¹⁸⁸Re-6D2 mAb in a mouse model (Dadachova et al. 2008). The high affinity constants of radiolabeled mAbs for their respective antigens are more important contributors to the efficacy of RIT than the number of binding sites (Dadachova et al. 2006b). The results obtained with ¹⁸⁸Re-11B11 (FIG. 2) were similar in magnitude to those observed with ¹⁸⁸Re-6D2 (FIG. 4A) despite the facts that the experiments were done at different times. Hence, the higher constant of ¹⁸⁸Re-11B11 relative to ¹⁸⁸Re-6D2 could have compensated for the fewer binding sites and contributed to comparable efficacy of treatment.

Interestingly, from Day 18 post-treatment, administration of “cold” 11B11 impaired tumor growth to some extent in comparison to untreated controls. The same effect was previously observed for “cold” 6D2 in both MNT1 and A2048-melanoma bearing mice. The histological examination of RIT-treated tumors revealed tumor infiltration by inflammatory cells. A possible explanation for this could be the induction of pro-inflammatory effects by murine IgM which is a potent activator of the complement system (Seynhaeve et al. 2006). The activation of complement elevates levels of some cytokines such as TNF-α which can result in inflammation (Seynhaeve et al. 2006). Thus, “cold” antibody bound to extracellular melanin at the tumor site provides synergistic immunological support for the cytocidal effects of RIT. Overall, the experiments with melanin-binding 11B11 mAb confirmed the results previously obtained with mAb 6D2 supporting the conclusion that the anti-tumor effects observed with these mAbs are due to their specificity for melanin.

Given the relative therapeutic equivalence of mAbs 6D2 and 11B11, further work focused on mAb 6D2 and evaluated the efficacy of combining chemotherapy and RIT with ¹⁸⁸Re-6D2. A rationale for combining chemotherapy and RIT was the premise that chemotherapy would kill some tumor cells and liberate intracellular melanin to provide more target for RIT. Dacarbazine (5-(3,3-dimethyl-1-triazeno)imidazole-4-carboxamide, DTIC) was selected as a chemotherapy agent. DTIC is considered the gold standard for treatment of patients with metastatic melanoma, despite only having a response rate of 15% to 20%, with most responses not being sustained (Sun and Schuchter, 2001). It also has been used previously in mouse melanoma models where 80 mg/kg/day of DTIC induced leucopenia in SCID mice (Halaschek-Wiener et al. 2003) whereas 50 mg/kg/day×5 days was relatively non-toxic and effectively slowed tumor growth (Fodstad et al. 1980). Histological analysis of A2058 tumors taken from mice treated for 5 days with 50 mg/kg DTIC demonstrated an increased amount of melanin in the extracellular space consistent with the notion that tumor death liberated intracellular melanin. A similar effect on the amount of extracellular melanin was noted in a prior study that analyzed histology after RIT (Dadachova et al. 2008).

The most obvious result of the therapy study was clear superiority of RIT over chemotherapy in the ability to control the tumor growth after day 15 and the superiority of combination therapy in the early stages of tumor growth. In patients with many different cancers including melanoma the early response to chemotherapy correlates with the significant decrease in ¹⁸F-FDG tumor uptake during PET and predicts the overall response to therapy (Strobel et al. 2008). Likewise, in tumor-bearing mice treated with chemotherapy the decrease in ¹⁸F-FDG tumor uptake in comparison with the baseline value was much less pronounced than in mice treated with RIT. Chemotherapy is expected to enhance RIT by increasing the amount of target melanin available to the melanin-binding antibody or peptide. In conclusion, pre-treatment of tumor-bearing mice with DTIC before administration of RIT made such treatment more effective at the early stages post-treatment than RIT alone.

### REFERENCES

U.S. Pat. No. 7,402,385, issued Jul. 22, 2008, Dadachova et al. Radiolabeled antibodies for treatment of tumors.
United States Patent Application Publication No. 2006/0039858, published Feb. 23, 2006, Dadachova et al. Radiolabeled antibodies and peptides for treatment of tumors.
Abbas A K, Lichtman A H, Pober J S. Cellular and Molecular Immunology, 4^{th} edition, W.B. Saunders Co., Philadelphia, 2000.
Buchsbaum D J 2000. Experimental radioimmunotherapy. Semin Radia. Oncol. 10: 156-67.
Busam K J, Hester K, Charles C, Sachs D L, Antonescu C R, Gonzalez S, Halpern A C. Detection of clinically amelanotic malignant melanoma and assessment of its margins by in vivo confocal scanning laser microscopy. Arch Dermatol 2001 July; 137 (7):923-9.
Cohen-Solal K A, Crespo-Carbone S M, Namkoong J, Mackason K R, Roberts K G, Reuhl K R, Chen S. Progressive appearance of pigmentation in amelanotic melanoma lesions. Pigment Cell Res 2002 August; 15 (4):282-9.
Dadachova E, Bryan R A, Apostolidis C, Morgenstern A, Zhang T, Moadel T, Torres M, Huang X, Revskaya E and Casadevall A. Interaction of radiolabeled antibodies with fungal cells and components of immune system in vitro and during radioimmunotherapy of experimental fungal infection. J. Infect. Dis., 193 (10):1427-1436 (2006b).
Dadachova E. and Casadevall A. Melanin as a potential target for radionuclide therapy of metastatic melanoma. Future Oncol. 1 (4), p. 541-549 (2005).
Dadachova E, Moadel T, Schweitzer A D, Bryan R A, Zhang T, Mints L, Revskaya E, Huang X, et al. Radiolabeled melanin-binding peptides are safe and effective in treatment of human pigmented melanoma in a mouse model of disease. Cancer Biother. Radiopharm. 2006(a) 21:117-129.
Dadachova E, Nosanchuk J D, Shi L et al. Dead cells in melanoma tumors provide abundant antigen for targeted delivery of ionizing radiation by a monoclonal antibody to melanin. Proc. Natl. Acad. Sci. USA 2004 101:14865-14870.
Dadachova E, Revskaya E, Sesay M A, Damania H, Boucher R, Sellers R S, Howell R C, Burns L, Thornton G B, Natarajan A, Mirick G R, Denardo S J, Denardo G L, Casadevall A. Pre-clinical evaluation and efficacy studies of a melanin-binding IgM antibody labeled with (188)Re against experimental human metastatic melanoma in nude mice. Cancer Biol. Ther. 7 (7). (2008). [Epub ahead of print].
Early P. J. and Sodee D. B. Principles and Practice of Nuclear Medicine, Mosby, 1995.
Fodstad O, Aass N, Pihl A. Response to chemotherapy of human, malignant melanoma xenografts in athymic, nude mice. Int J Cancer 1980; 25 (4):453-8.
Grin-Jorgensen C M, Rigel D S and Friedman R J The world-wide incidence of malignant melanoma. In: C. M. Balch, A. N. Houghton, G. W. Milton, A. J. Sober, and S. J. Soong (eds.), Cutaneous Melanoma, Ed. 2, pp. 27-39. Philadelphia: J.B. Lippincott Co., 1992.
Halaschek-Wiener J, Kloog Y, Wacheck V, Jansen B. Farnesyl thiosalicylic acid chemo-sensitizes human melanoma in vivo. J Invest Dermatol 2003; 120 (1):109-15.
Hoher M., Wohrle J, Wohlfrom M, Hanke H, Voisard R, Osterhues, H H, Kochs M, Reske S N, Hombach V, Kotzerke J. Intracoronary beta-irradiation with a liquid 188-Re-filled balloon: six-month results from a clinical safety and feasibility study. Circulation 101: 2355-2360, 2000.
Howell R. C., Revskaya E., Pazo V., Nosanchuk J. D., Casadevall A., Dadachova E. Phage display library derived peptides that bind to human tumor melanin as potential vehicles for targeted radionuclide therapy of metastatic melanoma. Bioconjugate Chem. 18:1739-48 (2007). Epub 2007 October 2.
Knapp, F. F. Jr. Rhenium-188—a generator-derived radioisotope for cancer therapy. Cancer Biother Radiopharm. 13: 337-349, 1998.
Klein M, Shibli N, Friedmann N, Thornton G B, Chisin R, Lotem M. Imaging of metastatic melanoma (MM) with a 188Rhenium (188Re)-labeled melanin binding antibody. J. Nucl. Med., 49 (Suppl 1): 52P (2008).
Lindmo T, Boven E, Cuttitta F, et al. Determination of the immunoreactive fraction of radiolabeled monoclonal antibodies by linear extrapolation to binding at infinite antigen excess. J Immunol Methods 1984; 72:77-89.
Liu T and Soong S J Epidemiology of malignant melanoma. Surg. Clin. N. Am., 76: 1205-1222, 1996.
Milenic D E, Brady E D and Brechbiel M W Antibody-targeted radiation cancer therapy. Nature Rev. Drug Discovery 2004 3:488-498.
Nosanchuk J D, Valadon P, Feldmesser M, Casadevall A. Melanization of Cryptococcus neoformans in murine infection. Mol Cell Biol. 19: 745-750, 1999.
Paganelli G., Zoboli S., Cremonesi M. et al Receptor-mediated radionuclide therapy with 90-Y-DOTA-D-Phe-Tyr³-Octreotide: Preliminary report in cancer patients. Cancer Biother. Radiopharm. 14: 477-483, 1999.
Palmedo H, Guhlke S, Bender H, Sartor J, Schoeneich G, Risse J, Grunwald F, Knapp F. F. Jr, Biersack H J. Dose escalation study with rhenium-188 hydroxyethylidene diphosphonate in prostate cancer patients with osseous metastases. Eur. J. Nucl. Med. 27: 123-130, 2000.
Rosas A L, Nosanchuk J D, Feldmesser M, Cox G M, McDade H C, and Casadevall A. Synthesis of polymerized melanin by Cryptococcus neoformans in infected rodents. Infect. Immun. 2000(a) 68: 2845-2853.
Rosas, A. L., Nosanchuk, J. D., Gomez, B. L., Edens, W. A., Henson, J. M., Casadevall, A., 2000(b). Isolation and serological analysis of fungal melanins. Journal of Immunological Methods. 244, 69-80.
Rigel D S Malignant melanoma: incidence issues and their effect on diagnosis and therapy in the 1990s. Mayo Clin. Proc., 72: 367-371, 1997.
Saha G B Fundamentals of Nuclear Pharmacy, Springer, 1997, New York, pp. 139-143
Schweitzer A. D., Rakesh V., Revskaya E., Datta A., Casadevall A., Dadachova E. Computational model predicts effective delivery of 188-Re-labeled melanin-binding antibody to the metastatic melanoma tumors with wide range of melanin concentrations. Melanoma Res. 17 (5):291-303 (2007).
Seynhaeve A L B, Vermeulen C E, Eggermont A M M, et al. Cytokines and vascular permeability: an in vitro study on human endothelial cells in relation to tumor necrosis factor-α-primed peripheral blood mononuclear cells. Cell Biochem Biophys 2006; 44:157-70.
Sgouros G, Ballangrud A M, Jurcic J G, McDevitt M R, Humm J L, Erdi Y E, Mehta B M, Finn R D, Larson S M and Scheinberg D A. Pharmacokinetics and dosimetry of an alpha-particle emitter labeled antibody: 213Bi-HuM195 (anti-CD33) in patients with leukemia. J. Nucl. Med. 40: 1935-1946. 1999.
Sharkey R M and Goldenberg D M Perspectives on cancer therapy with radiolabeled monoclonal antibodies. J Nucl Med 2005; 46 (Suppl 1):115S.
Srivastava S C and Dadachova E. Recent advances in radionuclide therapy. Semin. Nucl. Med. 2001 31: 330-341.
Strobel K, Dummer R, Steinert H C, Conzett K B, Schad K, Lago M P, Soyka J D, Veit-Haibach P, Seifert B, Kalff V. Chemotherapy response assessment in stage IV melanoma patients-comparison of (18)F-FDG-PET/CT, CT, brain MRI, and tumor marker S-100B. Eur J Nucl Med Mol Imaging. 2008 May 6. [Epub ahead of print].
Sun W, Schuchter L M. Metastatic melanoma. Curr Treat Options Oncol 2: 193-202, 2001.

1. A method for treating a melanin-containing melanoma in a subject which comprises (a) administering to the subject an agent or treatment that releases melanin from melanoma cells, and (b) then administering to the subject an amount of a radiolabeled anti-melanin antibody and/or radiolabeled anti-melanin peptide effective to treat the melanoma.

2. A method for imaging a melanin-containing melanoma in a subject which comprises (a) administering to the subject an agent or treatment that releases melanin from melanoma cells, and (b) then administering to the subject an amount of a radiolabeled anti-melanin antibody and/or radiolabeled anti-melanin peptide effective to image the melanoma.

3. The method of claim 1, wherein the agent or treatment that releases melanin from melanoma cells is one or more of chemotherapy, external beam radiation therapy, brachytherapy, immunotherapy, biological therapy, cellular therapy, gene therapy, or radionuclide therapy.

4. The method of claim 3, wherein the agent that releases melanin from melanoma cells is a drug that is cytotoxic for melanoma cells.

5. The method of claim 3, wherein the agent that releases melanin from melanoma cells is dacarbazine (5-(3,3-dimethyl-1-triazeno)imidazole-4-carboxamide, DTIC).

6. The method of claim 1, wherein the radiolabel is an alpha-emitting radioisotope.

7. The method of claim 6, wherein the alpha-emitting radioisotope is 213-Bismuth.

8. The method of claim 1, wherein the radiolabel is a beta-emitting radioisotope.

9. The method of claim 8, wherein the beta-emitting radioisotope is 188-Rhenium.

10. The method of claim 1, wherein the radiolabel is selected from the group consisting of a positron emitter; and an admixture of any of an alpha emitter, a beta emitter, and a positron emitter.

11. The method of claim 2, wherein the radiolabel is selected from the group consisting of 99m-Technetium, 111-Indium, 67-Gallium, 123-Iodine, 124-Iodine, 131-Iodine and 18-Fluorine.

12. The method of claim 1, wherein the radiolabel is administered at a dose between about 1-1000 mCi.

13. The method of claim 1, wherein a radiolabeled anti-melanin antibody is administered to the subject.

14. The method of claim 13, wherein the antibody is a monoclonal antibody.

15. The method of claim 13, wherein the antibody is an IgM antibody, an IgG antibody or an IgA antibody.

16. The method of claim 13, wherein the antibody is a humanized antibody or a chimeric antibody.

17. The method of claim 1, wherein a radiolabeled anti-melanin F(ab′)₂ fragment or a Fab′ fragment of a whole antibody is administered to the subject.

18. The method of claim 1, wherein a radiolabeled anti-melanin peptide is administered to the subject.

19. The method of claim 18, wherein the peptide is positively charged.

20. The method of claim 18, wherein the peptide is a decapeptide or a heptapeptide.

21. The method of claim 1, which comprises administering to the subject anti-melanin antibodies and/or anti-melanin peptides radiolabeled with a plurality of different radioisotopes.

22. The method of claim 21, wherein the radioisotopes are isotopes of a plurality of different elements.

23. The method of claim 21, wherein at least one radioisotope is a long range emitter and at least one radioisotope is a short range emitter.

24. The method of claim 23, wherein the long-range emitter is a beta emitter and the short range emitter is an alpha emitter.

25. The method of claim 24, wherein the beta emitter is 188-Rhenium and the alpha emitter is 213-Bismuth.

26. The method of claim 1, wherein the subject is a mammal.

27. The method of claim 26, wherein the mammal is a human.


SEQUENCE LISTING (22 sequences)

SEQ ID NO: 1 - protein, 10 aa, Artificial
  melanin-binding peptide 4B4
YERKFWHGRH

SEQ ID NO: 2 - protein, 10 aa, Artificial
  melanin-binding peptide
LHKLVRHGRW

SEQ ID NO: 3 - protein, 10 aa, Artificial
  melanin-binding peptide
YLRRHTHVFW

SEQ ID NO: 4 - protein, 10 aa, Artificial
  melanin-binding peptide
KKHSHYWVRY

SEQ ID NO: 5 - protein, 10 aa, Artificial
  melanin-binding peptide
EFGTRHMRHR

SEQ ID NO: 6 - protein, 10 aa, Artificial
  melanin-binding peptide
YRHHAHGGRG

SEQ ID NO: 7 - protein, 10 aa, Artificial
  melanin-binding peptide
RKKWHGWTRW

SEQ ID NO: 8 - protein, 10 aa, Artificial
  melanin-binding peptide
PKWRHGYTRF

SEQ ID NO: 9 - protein, 10 aa, Artificial
  melanin-binding peptide
RHGTVKHARH

SEQ ID NO: 10 - protein, 10 aa, Artificial
  melanin-binding peptide
RRHWHPPVQI

SEQ ID NO: 11 - protein, 10 aa, Artificial
  melanin-binding peptide
EAYKRRWHWP

SEQ ID NO: 12 - protein, 10 aa, Artificial
  melanin-binding peptide
RWPKRHLSGH

SEQ ID NO: 13 - protein, 10 aa, Artificial
  melanin-binding peptide
SRVPFRHYHH

SEQ ID NO: 14 - protein, 10 aa, Artificial
  melanin-binding peptide
RRPEHTKARW

SEQ ID NO: 15 - protein, 10 aa, Artificial
  melanin-binding peptide
WRAFLPRWHA

SEQ ID NO: 16 - protein, 10 aa, Artificial
  melanin-binding peptide
WNRGWRWWMG

SEQ ID NO: 17 - protein, 10 aa, Artificial
  melanin-binding peptide
GFFWKWRIGR

SEQ ID NO: 18 - protein, 10 aa, Artificial
  melanin-binding peptide
HIRWKGHISW

SEQ ID NO: 19 - protein, 5 aa, Artificial
  melanin-binding peptide
XXXXH

SEQ ID NO: 20 - protein, 7 aa, Artificial
  melanin-binding peptide
NPNWGPR

SEQ ID NO: 21 - protein, 7 aa, Artificial
  melanin-binding peptide
HTTHHRN

SEQ ID NO: 22 - protein, 7 aa, Artificial
  melanin-binding peptide
TTHQFPF